(19) United States
(12) Patent Application Publication
(10) Pub. No.: US 2015/0072427 A1
(43) Pub. Date: Mar. 12, 2015

(54) Dedifferentiation and Reprogramming of Cells

(71) Applicant: Stemnion, Inc., Pittsburgh, PA (US)

(72) Inventor: George L. Sing, New York, NY (US)

(73) Assignee: Stemnion, Inc., Pittsburgh, PA (US)

(21) Appl. No.: 14/548,581

(22) Filed: Nov. 20, 2014

Related U.S. Application Data
(63) Continuation of application No. 13/138,193, filed on Jul. 18, 2011.
(60) Provisional application No. 61/269,975, filed on Jul. 1, 2009.
(60) Provisional application No. 61/205,235, filed on Jan. 20, 2009.

(51) Int. Cl. C12N 5/074; C12N 5/073

(52) U.S. Cl. CPC C12N 5/0696; C12N 5/0605; C12N 2506/025
USPC 435/377

(57) ABSTRACT

The invention is directed to methods for reprogramming somatic cells to a less differentiated state. In particular, the invention is directed to methods for reprogramming amnion epithelial cells (AEC) including amnion-derived cells (ADC) and Amnion-derived Multipotent Progenitor cells (AMP cells) to a less differentiated state. The invention is further directed to compositions comprising reprogrammed AEC, ADC and AMP cells, and uses thereof.

### CROSS-REFERENCE TO RELATED APPLICATIONS

This application claims priority under 35 USC §119(e) of U.S. Provisional Application Nos. 61/269,975, filed Jul. 1, 2009, and 61/205,235, filed Jan. 20, 2009, the entireties of which are incorporated herein by reference.

### FIELD OF THE INVENTION

The field of the invention is directed to methods for reprogramming somatic cells to a less differentiated status. In particular, the field of the invention is directed to methods for reprogramming amnion epithelial cells (AEC), including amnion-derived cells (ADC) and Amnion-derived Multipotent Progenitor cells (AMP cells), to a less differentiated status. The field is further directed to compositions comprising reprogrammed AEC, ADC and AMP cells, and uses thereof.

### DESCRIPTION OF RELATED ART

Yamanaka, S. (Philos Trans R Soc Lond B Biol Sci 2008 363(1500):2079-87) reviews molecular mechanisms of and known methods of inducing pluripotency in somatic cells.

Yamanaka, S. (Cell Prolif 2008 Suppl 1:51-6) describes induction of pluripotent stem cells from mouse fibroblasts by four transcription factors.

Okita, K., et al., (Science 2008 322(5903)949-53, Epub 2008 Oct. 9) describe generation of mouse induced pluripotent stem cells with out viral vectors.

Park, I. H., et al., (Nature 451:141-6, 2008) describe reprogramming of human somatic cells to pluripotency with defined factors.

Yu, J., et al., (Science 318:1917-20, 2007) describe induced pluripotent stem cell lines derived from human somatic cells.

Takahashi, K., et al., (Nat Protoc 2007 2(12):3081-9) describe induction of pluripotent stem cells from fibroblast cultures.

Oliveri, R. S. (Regen Med 2007 2(5):795-816) reviews epigenetic dedifferentiation of somatic cells into pluripotency.

Alberio, R., et al., (Reproduction 2006 132(5):709-20) reviews reprogramming somatic cells into stem cells.

U.S. Publication No. 20080280362, published Nov. 13, 2008, describes methods for reprogramming somatic cells.

### BACKGROUND OF THE INVENTION

The differentiation status of cells is a continuous spectrum, with the terminally differentiated state at one end and de-differentiated state (the pluripotent state) at the other end. Reprogramming encompasses any movement of the differentiation status of a cell along the spectrum toward a less-differentiated state. For example, reprogramming includes reversing a multipotent cell back to a pluripotent cell or reversing a terminally differentiated cell back to either a multipotent cell or a pluripotent cell.

Much research is directed to developing methods for reprogramming cells to a less differentiated status. Such methods include but are not limited to viral-induced reprogramming through the introduction of pluripotency genes into cells via viral vectors, contacting cells with chemical agents (i.e. demethylating agents) that alter chromatin structure and consequently differentiation status, nuclear transfer methodologies, and contacting cells with unique media and matrix combinations that effect dedifferentiation. Most of the methods described thus far have been successful to at least some degree in most cells tested, although some, for example viral-induced dedifferentiation and reprogramming, do have associated risks such as teratoma formation which make the clinical application of these reprogrammed cells not feasible at this time.

### SUMMARY OF THE INVENTION

The invention is directed to methods for reprogramming somatic cells to a less differentiated status. In particular, the field of the invention is directed to methods for reprogramming amnion epithelial cells (AEC), including amnion-derived cells (ADC) and Amnion-derived Multipotent Progenitor cells (AMP cells), to a less differentiated status. In accordance with the methods of the invention, the AEC, ADC and/or AMP cells, are contacted with a candidate agent capable of effecting reprogramming of the cells to a less differentiated status. Dedifferentiated cells are then selected and assessed for pluripotency characteristics (i.e., teratoma formation, embryoid body formation, expression of pluripotent cell markers, lack of expression of differentiation markers, etc.). The presence of at least a subset of pluripotency characteristics in the cells indicates that the agent is capable of reprogramming the cells to a less differentiated status. The invention is further directed to compositions comprising the reprogrammed cells, as well as uses of the reprogrammed cells. Once reprogrammed, the cells are termed AEC^{R}, ADC^{R} and AMP^{R} cells. AEC^{R}, ADC^{R} and AMP^{R} cells can be treated with various differentiation media, agents, condition, etc., to induce them to differentiate down any cellular pathway. For example, the AEC^{R}, ADC^{R} and/or AMP^{R} can be exposed to conditions known to effect neural differentiation, pancreatic differentiation, hematopoietic differentiation, and the like. The advantage of using AEC, ADC and/or AMP cells is that the cells are obtained from a non-controversial source, the normally discarded placenta, and therefore do not possess the assorted ethical, religious or political issues that are associated with ES cells. In addition, AEC, ADC and/or AMP cells may already express one or more pluripotency genes (i.e. Oct4), which may aid in the dedifferentiation of these cells.

Accordingly, a first aspect of the invention is a method of reprogramming amnion epithelial cells to a less differentiated state comprising contacting the cells with an agent capable of effecting such reprogramming. In one embodiment the amnion epithelial cells are amnion-derived cells or AMP cells. In another embodiment the amnion epithelial cells are human amnion epithelial cells. In still another embodiment the agent is a pluripotency gene. And in a specific embodiment the pluripotency gene is Oct4, Sox2, Klf4, c-Myc, nanog, Lin28, or Stella. Other specific embodiments are ones in which the pluripotency gene is one of Oct4, Sox2, Klf4, c-Myc, nanog, Lin28, or Stella; the pluripotency gene is two of Oct4, Sox2, Klf4, c-Myc, nanog, Lin28, or Stella; the pluripotency gene is three of Oct4, Sox2, Klf4, nanog, Lin28, Stella or c-Myc; the pluripotency gene is four of Oct4, Sox2, Klf4, c-Myc, nanog, Lin28, or Stella; the pluripotency gene is five of Oct4, Sox2, Klf4, c-Myc, nanog, Lin28, or Stella; the pluripotency gene is six of Oct4, Sox2, Klf4, c-Myc, nanog, Lin28, or Stella; or the pluripotency gene is all of Oct4, Sox2, Klf4, c-Myc, nanog, Lin28, or Stella.

In another embodiment, the pluripotency gene is delivered to the amnion epithelial cell by retrovirus-mediated transfection, lentivirus-mediated transfection, adenovirus-mediated DNA transfection or non-viral-mediated DNA transfection. In still another embodiment the agent is a demethylating agent or a deacetylation agent. In a specific embodiment the demethylating agent is a 5-aza-cytidine or 5-azadeoxycytidine. In another specific embodiment the deacetylation agent is trichostatin A, trapoxin B, depsipeptides, benzamides, electrophilic ketones, phenylbutyrate or valproic acid. In another embodiment the less differentiated state is totipotency or pluripotency.

A second aspect of the invention is a dedifferentiated cell made by the method of the first aspect.

A third aspect of the invention is a method of treating a disease or disorder in a subject in need thereof comprising transplanting the dedifferentiated cell of the second aspect into the subject.

A fourth aspect of the invention is a composition comprising AEC^{R}, ADC^{R} or AMP^{R} cells, or a combination thereof, wherein the cells exhibit pluripotency characteristics. In one embodiment the pluripotency characteristic is expression of one or more ES cell markers. In a specific embodiment the ES cell markers are Oct4, SSEA1, SSEA3, SSEA4, elevated Alkaline Phosphatase levels, nestin, AC133, Tcf4 or Cdx1. In still another embodiment the pluripotency characteristic is expression of pluripotency genes. And in a particular embodiment the pluripotency genes are one or more of Oct4, Sox2, Klf4, m-Myc, nanog, Lin28, or Stella. In yet another embodiment the pluripotency characteristic is the ability to differentiate into any cell type in body. And in another embodiment the pluripotency characteristic is the ability to form embryoid bodies. In still another embodiment the pluripotency characteristic is having the capacity for self-renewal.

A fifth aspect of the invention is a composition comprising AEC^{R}, ADC^{R} or AMP^{R} cells wherein the cells are capable of forming any cell type which arises from the endoderm. In particular embodiments, the cell type which arises from the endoderm is a stomach cell, colon cell, liver cell, pancreas cell, urinary bladder cell, lining of the urethra cell, epithelial parts of the trachea cell, lung cell, pharynx cell, thyroid cell, parathyroid cell, or intestinal cell.

A sixth aspect of the invention is a composition comprising AEC^{R}, ADC^{R} or AMP^{R} cells wherein the cells are capable of forming any cell type which arises from the mesoderm. In particular embodiments, the cell type which arises from the mesoderm is a skeletal muscle cell, skeletal cell, dermal cell, connective tissue cell, urogenital system cell, heart cell, blood cell, lymph cells, or spleen cell.

A seventh aspect of the invention is a composition comprising AEC^{R}, ADC^{R} or AMP^{R} cells wherein the cells are capable of forming any cell type which arises from the ectoderm. In particular embodiments, the cell type which arises from the ectoderm is a central nervous system cell, lens cell, cranial and sensory nerve cell, motor nerve cell, ganglion cell, pigment cell, head connective tissue cell, epidermal cell, hair cell, or mammary gland cell.

### Definitions

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “marker” means any molecule characteristic of a cell or in some cases of a specific cell type.

As used herein, the term “protein marker” means any protein molecule characteristic of a cell or in some cases of a specific cell type. Protein markers may be located on the cell membrane, may be intracellular or may be secreted from the cell.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent stem cells” shall have the following meaning In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

The term “self-renewal” as used herein means a cell or population of cells having the ability to go through numerous cycles of cell division while maintaining the undifferentiated state.

The term “somatic cells”, as used herein, also includes adult stem cells. An adult stem cell is a cell that is capable of giving rise to all cell types of a particular tissue. Exemplary adult stem cells include hematopoietic stem cells, neural stem cells, and mesenchymal stem cells.

The term “pluripotency gene”, as used herein, refers to a gene that is associated with pluripotency. The expression of a pluripotency gene is typically restricted to pluripotent stem cells, and is crucial for the functional identity of pluripotent stem cells.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cytokine secreting cells” or “ECS cells” means a population of cells derived from the extraembryonic tissue which have the characteristics of secreting a unique combination of physiologically relevant cytokines in a physiologically relevant temporal manner into the extracellular space or into surrounding culture media and which have not been cultured in the presence of any non-human animal-derived components, making them and cell products derived from them suitable for human clinical use. In one embodiment, the ECS cells secrete at least one cytokine selected from VEGF, angiogenin, PDGF and TGFβ32 and at least one MMP inhibitor selected from TIMP-1 and TIMP-2. In another embodiment, the ECS cells secrete more than one cytokine selected from VEGF, angiogenin, PDGF and TGFβ32 and more than one MMP inhibitor selected from TIMP-1 and TIMP-2. In a preferred embodiment, the ECS cells secrete the cytokines VEGF, angiogenin, PDGF and TGFβ2 and the MMP inhibitors TIMP-1 and TIMP-2. The physiological range of the cytokine or cytokines in the unique combination is as follows: ˜5-16 ng/mL for VEGF, ˜3.5-4.5 ng/mL for angiogenin, ˜100-165 pg/mL for PDGF, ˜2.5-2.7 ng/mL for TGFβ2, ˜0.68 μg mL for TIMP-1 and ˜1.04 μg/mL for TIMP-2. ECS cells may be selected from populations of cells and compositions described in this application and in US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179, the contents of which are incorporated herein by reference in their entirety.

As used herein, the term “Amnion-derived Multipotent Progenitor cell” or “AMP cell” means a specific population of ECS cells that are epithelial cells derived from the amnion. In addition to the characteristics described above for ECS cells, AMP cells have the following characteristics. They have not been cultured in the presence of any non-human animal-derived components, making them and cell products derived from them suitable for human clinical use. They grow without feeder layers, do not express the protein telomerase and are non-tumorigenic. AMP cells do not express the hematopoietic cell markers CD34 and CD45 protein. The absence of CD34 and CD45 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are derived, will not react with antibodies to the stem/progenitor cell markers c-kit (CD 117) and Thy-1 (CD90). AMP cells will not react with antibodies to the stem/progenitor cell markers c-kit (CD117). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells. AMP cells have previously been described as “amnion-derived cells” (see U.S. Provisional Application Nos. 60/666,949, 60/699,257, 60/742,067, U.S. Provisional Application Nos. 60/813,759, U.S. application Ser. No. 11/333,849, U.S. application Ser. No. 11/392,892, and PCTUS06/011392, each of which is incorporated herein in its entirety).

By the term “animal-free” when referring to compositions, growth conditions, culture media, etc. described herein, is meant that no non-human animal-derived components, such as animal-derived serum, protein, carbohydrate, lipid, nucleic acid, vitamin, co-enzyme, etc., are used in the preparation, growth, culturing, expansion, or formulation of the composition or process. Only human-derived components may be used in the preparation, growth, culturing, expansion, or formulation of the composition or process.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such primary cells are subcultured, each round of subculturing is referred to as a passage. As used herein, “primary culture” means the freshly isolated cell population.

As used herein, the terms “a” or “an” means one or more; at least one.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 2007, “Current Protocols in Molecular Biology” Volumes I-IV; Celis, ed., 2005, “Cell Biology: A Laboratory Handbook” Volumes I-III; Coligan, ed., 2007, “Current Protocols in Immunology”; Gait ed., 1984, “Oligonucleotide Synthesis”; Hames & Higgins eds., 1991, “Nucleic Acid Hybridization”; Hames & Higgins, eds., 1985, “Transcription And Translation: A Practical Approach”; Freshney, ed., 2006, “Animal Cell Culture” 2^{nd} Ed.; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Obtaining and Culturing of Cells

Various methods for isolating cells from the amnion of the placenta, which may then be used to obtain AEC, ADC and AMP cells and subsequently produce the dedifferentiated and reprogrammed cells of the instant invention, are described in the art (see, for example, US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179).

In particular, AMP cell compositions are prepared using the steps of a) recovery of the amnion from the placenta, b) dissociation of the cells from the amniotic membrane, c) culturing of the cells in a basal medium with the addition of a naturally derived or recombinantly produced human protein; d) selecting AMP cells from the cell culture, and optionally e) further proliferation of the cells, optionally using additional additives and/or growth factors. Details are contained in US Publication No. 2006-0222634-A1, which is incorporated herein by reference.

AMP cells are cultured as follows: The AMP cells are cultured in a basal medium. Such medium includes, but is not limited to, Epilife (Cascade Biologicals), Opti-pro, VP-SFM, IMDM, Advanced DMEM, K/O DMEM, 293 SFM II (all made by Gibco; Invitrogen), HPGM, Pro 293S-CDM, Pro 293A-CDM, UltraMDCK, (all made by Cambrex), Stemline I and Stemline II (both made by Sigma-Aldrich), DMEM, DMEM/F-12, Ham's F12, M199, and other comparable basal media. Such media may either contain human protein or be supplemented with human protein. As used herein a “human protein” is one that is produced naturally or one that is produced using recombinant technology. “Human protein” also is meant to include a human fluid or derivative or preparation thereof, such as human serum or amniotic fluid, which contains human protein. Details on this procedure are contained in US Publication No. 2006-0222634-A1, which is incorporated herein by reference.

In a most preferred embodiment, the cells are cultured using a system that is free of animal products to avoid xeno-contamination. In this embodiment, the culture medium is IMDM, Stemline I or II, Opti-pro, or DMEM, with human albumin added up to concentrations of 10%. The invention further contemplates the use of any of the above basal media wherein animal-derived proteins are replaced with recombinant human proteins and animal-derived serum, such as BSA, is replaced with human albumin. In preferred embodiments, the media is serum-free in addition to being animal-free. Details on this procedure are contained in US Publication No. 2006-0222634-A1, which is incorporated herein by reference.

In alternative embodiments, where the use of non-human serum is not precluded, such as for in vitro uses, the culture medium may be supplemented with serum derived from mammals other than humans, in ranges of up to 40%.

Genes and DNA Constructs

In accordance with the present invention, AEC, ADC and/or AMP cells may be genetically manipulated such that they comprise one or more pluripotency gene(s) (i.e., Oct4, Sox2, Klf4, c-Myc, nanog, Lin28, or Stella) linked to DNA encoding a selectable marker in such a manner that the expression of the selectable marker substantially matches the expression of the pluripotency gene. In one embodiment, the AEC, ADC and/or AMP cells comprise a first pluripotency gene linked to DNA encoding a first selectable marker in such a manner that the expression of the first selectable marker substantially matches the expression of the first pluripotency gene. The AEC, ADC and/or AMP cells may also be engineered to comprise any number of pluripotency genes, each respectively linked to a distinct selectable marker. The AEC, ADC and/or AMP cells may also be engineered to have one or more pluripotency gene expressed as a transgene under an inducible promoter. In a preferred embodiment, the AEC, ADC and/or AMP cells are genetically manipulated to comprise the Oct4, Sox2, Klf4, and c-Myc pluripotency genes.

The selectable marker may be linked to an appropriate pluripotency gene such that the expression of the selectable marker substantially matches the expression of the pluripotency gene i.e., the selectable marker and the pluripotency gene are co-expressed, although it is not necessary that their relative expression levels be the same or even similar. It is only necessary that the AEC, ADC and/or AMP cells in which a pluripotency gene is activated will also express the selectable marker at a level sufficient to confer a selectable phenotype on the reprogrammed cells. Skilled artisans are familiar with selectable markers commonly used in genetic engineering strategies.

The DNA encoding a selectable marker may be inserted downstream from the end of the open reading frame (ORF) encoding the desired pluripotency gene, anywhere between the last nucleotide of the ORF and the first nucleotide of the polyadenylation site. An internal ribosome entry site (IRES) may be placed in front of the DNA encoding the selectable marker. Alternatively, the DNA encoding a selectable marker may be inserted anywhere within the ORF of the desired pluripotency gene, downstream of the promoter, with a termination signal. An internal ribosome entry site (IRES) may be placed in front of the DNA encoding the selectable marker. Skilled molecular biologists recognize that many other suitable constructs are possible and all are contemplated by the methods of the invention.

Methods for Reprogramming AEC, ADC and/or AMP Cells

In general, the methods for reprogramming AEC, ADC and/or AMP cells comprise treating the cells with an agent capable of effecting dedifferentiation and reprogramming. Such treatment may involve contacting the cells with an agent which alters chromatin structure (i.e., a demethylating agent), or may involve transfecting the cells with one or more pluripotency gene(s) (as described above), or both. The above two treatments may be concurrent or sequential. Reprogrammed AEC, ADC and AMP cells (termed AEC^{R}, ADC^{R} and AMP^{R} cells) are identified by selecting for cells that express the appropriate selectable marker. In addition, AEC^{R}, ADC^{R} and/or AMP^{R} cells are assessed for the presence of pluripotency characteristics. The presence of pluripotency characteristics indicates that the AEC, ADC and/or AMP cells have been reprogrammed to a pluripotent status.

The term “pluripotency characteristics”, as used herein, refers to many characteristics associated with pluripotency, including but not limited to, for example, the ability to differentiate into all types of cells and having a gene expression pattern distinct for a pluripotent cell, including for example expression of pluripotency genes (i.e., Oct4, Sox2, Klf4, c-Myc, nanog, Lin28, or Stella), expression of other ES cell markers (i.e., SSEA-1, SSEA-3, SSEA-4, elevated Alkaline Phosphatase levels, nestin, AC133, Tcf4 or Cdx1), lack of expression of differentiation markers, in some instances teratoma formation, embryoid body formation (i.e., aggregates of cells derived from embryonic stem cells), etc. Self-renewing capacity, marked by induction of telomerase activity, is another pluripotency characteristic that can be assessed. Functional assays of the AEC^{R}, ADC^{R} and/or AMP^{R} cells may be performed by introducing the cells into blastocysts and determining whether the cells are capable of forming some cell types, wherein they are multipotent; if the AEC^{R}, ADC^{R} and/or AMP^{R} cells are capable of forming all cell types of the body including germ cells, they are pluripotent.

AEC, ADC and/or AMP cells may be reprogrammed to gain a complete set of the pluripotency characteristics. Alternatively, AEC, ADC and/or AMP cells may be reprogrammed to gain only a subset of the pluripotency characteristics.

Expression of an exogenous pluripotency gene may occur in several ways. In one embodiment, the exogenously introduced pluripotency gene may be expressed from a chromosomal locus different from the endogenous chromosomal locus of the pluripotency gene. Such chromosomal locus may be a locus with open chromatin structure and contain a gene dispensable for the cell. An exemplary chromosomal locus is the human ROSA 26 locus (see, for example, Irion, et al., Nature Biotechnology 25, 1477-1482 (2007)). The exogenously introduced pluripotency gene may be expressed from an inducible promoter such that their expression can be regulated as desired. The term “inducible promoter”, as used herein, refers to a promoter that, in the absence of an inducer (such as a chemical and/or biological agent), does not direct expression, or directs low levels of expression of an operably linked gene (including cDNA), and, in response to an inducer, its ability to direct expression is enhanced. Skilled artisans are familiar with inducible promoters and their application.

In an alternative embodiment, the exogenously introduced pluripotency gene may be transiently transfected into AEC, ADC and/or AMP cells, either individually or as part of a cDNA expression library, such library prepared from pluripotent cells. The cDNA library is prepared by conventional techniques familiar to skilled artisans.

Several agents may be used in the methods which may cause chromatin to take on a more open structure, which is more permissive for gene expression. For example, DNA methylation and histone acetylation are two known events that alter chromatin toward a more closed structure. Loss of methylation by genetic deletion of the DNA methylation enzyme Dnmt1 in fibroblasts results in reactivation of endogenous Oct4 gene. See J. Biol. Chem. 277: 34521-30, 2002; and Bergman and Mostoslaysky, Biol. Chem. 1990. Thus, DNA methylation inhibitors and histone deacetylation inhibitors are two classes of agents that may be used in the methods of the invention. Exemplary demethylation agents include 5-aza-cytidine or 5-azadeoxycytidine and deacetylation agents include trichostatin A, trapoxin B, depsipeptides, benzamides, electrophilic ketones, phenylbutyrate or valproic acid.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of AMP Cell Compositions

Recovery of AMP cells—Amnion epithelial cells were dissociated from starting amniotic membrane using the dissociation agent PXXIII. The average weight range of an amnion was 18-27 g. The number of cells recovered per g of amnion was about 10-15×10⁶ for dissociation with PXXIII.

Method of selecting AMP cells: Amnion epithelial cells were isolated from the amnion and frozen in liquid nitrogen. Once thawed, the cells were plated and after ˜2-3 days in culture non-adherent cells were removed and the adherent cells were kept. The adherent cells represent about 30% of the plated cells. This attachment to plastic tissue culture vessel is the selection method used to obtain the desired population of AMP cells. Adherent and non-adherent cells appear to have a similar cell surface marker expression profiles but the adherent AMP cells have greater viability and are the desired population of cells. Selected AMP cells were cultured until they reached ˜120,000-300,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reach ˜120,000-300,000 cells/cm², they were collected and cryopreserved. This collection time point is called p0.

### Example 2

### DNA Constructs for Introducing Pluripotency Genes into Cells

DNA constructs containing pluripotency genes are constructed. The constructs may be transfection plasmids or they may be viral vectors.

The DNA constructs may contain one pluripotency gene (i.e., any of one Oct4, Sox2, Klf4, c-Myc, nanog, Lin28, or Stella) or they may contain one, two, three, four, five, six or seven pluripotency genes. Many combinations of the different pluripotency genes is also contemplated. For example, a DNA construct may contain Oct4 and Sox2; Klf4 and Stella; Oct4, Sox2 and Klf4, etc. A preferred DNA construct contains Oct4, Sox2, Klf4, and c-Myc. Any and all combinations of pluripotency genes in DNA constructs are contemplated by the invention.

To construct the DNA constructs, the cDNAs for the pluripotency genes can be obtained from various sources. For example, the cDNAs may be purchased from GeneCopoeia, Inc., 18520 Amaranth Drive, Germantown, Md., 20874 (www.genecopoeia.com) using the following product IDs: Oct4 Product ID T2820; Sox2 Product ID T2547; Klf4 Product ID Q0453; nanog Product ID W2005; Stella Product ID Y4255. c-Myc can be obtained from OriGene Technologies, Inc., 6 Taft Court, Suite 100, Rockville, Md., 20850 (www.origene.com) catalog # SC107923.

Viral vectors can be obtained from several sources as well. For example, lentiviral packaging kits can be obtained from GeneCopoeia, Inc., 18520 Amaranth Drive, Germantown, Md., 20874 (www.genecopoeia.com), Product No. PLv-PK-01. Retroviral packaging kits can be obtained from Fischer Scientific, Inc., 2000 Park Lane Drive, Pittsburgh, Pa. 15275, Catalog No. 6160 or 6161. Adenoviral expression kits can be obtained from Invitrogen, Inc., Carlsbad, Calif. 92008 (www.invitrogen.com) SKU #K4930-00.

Skilled artisans are familiar with standard molecular biology protocols for the construction of DNA constructs. Any standard methodology for the introduction of DNA into cells is suitable for use in the methods of the invention, including calcium phosphate precipitation, lipofection, electroporation, infection with viral vectors, etc.

### Example 3

### Culture Method for Producing Pluripotent Cells or Maintaining Pluripotency of Cells

Specific culture methods are suitable for producing pluripotent cells. For example, the method described by Brons, et al (Nature 2007, 448:191-195) or the method described by Tesar et al (Nature 2007, 448:196-199) is suitable for producing pluripotent cells or for maintaining pluripotency of cells. Cells suitable for use in such methods include the AEC^{R}, ADC^{R} and/or AMP^{R} cells described herein, or other reprogrammed or induced pluripotent cells known to skilled artisans, for examples, those described by Yamanaka, S. (Philos Trans R Soc Lond B Biol Sci 2008 363(1500):2079-87); Yamanaka, S. (Cell Prolif 2008 Suppl 1:51-6), Okita, K., et al., (Science 2008 322(5903)949-53, Epub 2008 Oct 9); Park, I. H., et al., (Nature 451:141-6, 2008), Yu, J., et al., (Science 318:1917-20, 2007); Takahashi, K., et al., (Nat Protoc 2007 2(12):3081-9); Oliveri, R. S. (Regen Med 2007 2(5):795-816); Alberio, R., et al., (Reproduction 2006 132(5):709-20); and U.S. Publication No. 20080280362, each of which is incorporated herein by reference. Naturally occurring pluripotent cells such as ES cells and cells derived from a pre-implantation embryo, are also suitable for use in the methods.

### Example 4

### Analyzing Cells for Pluripotency

Any number of assays and analyses are used to assess the pluripotency of the AEC^{R}, ADC^{R} and/or AMP^{R} cells. For example, RT-PCR is performed to detect expression of pluripotency genes (i.e., Oct4, Sox2, Klf4, c-Myc, nanog, Lin28, or Stella). FACS is performed to detect the expression of cell surface markers (i.e., SSEA-1, SSEA-3, SSEA-4). AEC^{R}, ADC^{R} and/or AMP^{R} cells are injected into SCID mice to look for teratoma formation. The AEC^{R}, ADC^{R} and/or AMP^{R} cells are cultured to detect embryoid body formation. Self-renewing capacity, marked by induction of telomerase activity, is assessed by RT-PCR. Functional assays of the AEC^{R}, ADC^{R} and/or AMP^{R} cells is performed by introducing the cells into blastocysts and determining whether the cells are capable of forming some cell types.

### Example 5

### Uses of Reprogrammed Cells

AEC^{R}, ADC^{R} and AMP^{R} cells are treated with various differentiation media, agents, conditions, etc., to induce them to differentiate down any cellular pathway. For example, the AEC^{R}, ADC^{R} and/or AMP^{R} are exposed to conditions known to effect differentiation of cells arising from all three primary germ layers, the endoderm, mesoderm and ectoderm. The endoderm forms the stomach, the colon, the liver, the pancreas, the urinary bladder, the lining of the urethra, the epithelial parts of trachea, the lungs, the pharynx, the thyroid, the parathyroid, and the intestines. The mesoderm forms: skeletal muscle, the skeleton, the dermis of skin, connective tissue, the urogenital system, the heart, blood (lymph cells), and the spleen. The ectoderm forms: the central nervous system, the lens of the eye, cranial and sensory, the ganglia and nerves, pigment cells, head connective tissues, the epidermis, hair, and mammary glands.

Such differentiated cells are then used to treat various conditions, for example, diabetes, heart disease, nervous system disease, etc.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

1.-33. (canceled)

34. A composition comprising reprogrammed Amnion-derived Multipotent Progenitor (AMP^{R}) cells, wherein the cells exhibit pluripotency characteristics.

35. The composition of claim 34 wherein the pluripotency characteristics are expression of one or more of the embryonic stem (ES) cell markers selected from the group consisting of Oct4, SSEA1, SSEA3, SSEA4, elevated Alkaline Phosphatase levels, nestin, AC133, Tcf4, and Cdx1.

36. The composition of claim 34 wherein the pluripotency characteristics are expression of one or more pluripotency genes.

37. The composition of claim 36 wherein the pluripotency genes are selected from the group consisting of one or more of Oct4, Sox2, Klf4, m-Myc, nanog, Lin28, and Stella.

38. The composition of claim 34 wherein the pluripotency characteristics are selected from the group consisting of the ability to differentiate into any cell type in the body, the ability to form embryoid bodies, and the ability for self-renewal.

39. A composition comprising AMP^{R} cells, wherein the cells are capable of differentiating into any cell type which arises from the endoderm, mesoderm, or ectoderm.

40. The composition of claim 39 wherein the cell type which arises from the endoderm is selected from the group consisting of a stomach cell, colon cell, liver cell, pancreas cell, urinary bladder cell, lining of the urethra cell, epithelial parts of the trachea cell, lung cell, pharynx cell, thyroid cell, parathyroid cell, and intestinal cell; wherein the cell type which arises from the mesoderm is selected from the group consisting of a skeletal muscle cell, skeletal cell, dermal cell, connective tissue cell, urogenital system cell, heart cell, blood cell, lymph cell, and spleen cell; and wherein the cell type which arises from the ectoderm is selected from the group consisting of a central nervous system cell, lens cell, cranial and sensory nerve cell, motor nerve cell, ganglion cell, pigment cell, head connective tissue cell, epidermal cell, hair cell, and mammary gland cell.
